(19) United States
(12) Patent Application Publication
(10) Pub. No.: US 2003/0190688 A1
(43) Pub. Date: Oct. 9, 2003

(54) Methods for detecting BCR-ABL signaling activity in tissues using phospho-specific antibodies

(75) Inventors: Katherine Crosby, Middleton, MA (US); Bradley L. Smith, Marblehead, MA (US); Valerie L. Goss, Gloucester, MA (US)

Correspondence Address: James Gregory Cullem, Esq., Intellectual Property Counsel, CELL SIGNALING TECHNOLOGY, INC., 166B Cummings Center, Beverly, MA, 01915, US

(73) Assignee: Cell Signaling Technology, Inc.

(21) Appl. No.: 10/408,486

(22) Filed: Apr. 7, 2003

Related U.S. Application Data
(60) Provisional application No. 60/370,554, filed on Apr. 5, 2002.

(51) Int. Cl. G01N 33/574

(52) U.S. Cl. 435/7.23

(57) ABSTRACT

The invention provides novel reagents and methods for detecting BCR-ABL or c-Abl kinase activity, and/or Abl signaling pathway activation in a cell or tissue, and discloses novel biomarkers relevant to Abl-mediated disease progression and therapeutic responsiveness, and provides predictive and detection methods based on the same. Phosphorylated BCR-ABL (Tyr245) and/or c-Abl (Tyr245), BCR-ABL (Tyr735) and/or c-Abl (Tyr735), Bcr (Tyr177), CRKL (Tyr207), Gab1 (Tyr627), PYK2 (Tyr402), Tyk2 (Tyr1054/1055), SHP2 (Tyr580), ERK1/2 (Thr202/Tyr204) and MEK1/2 (Ser217/221) have now been identified as relevant biomarkers of c-Abl pathway-mediated disease, and phospho-specific antibodies to these targets are provided. Kits for carrying out the methods of the invention are also provided.

### RELATED APPLICATIONS

This application claims priority to U.S. S No. 60/370,554, filed Apr. 5, 2002, now abandoned, the disclosure of which is hereby incorporated by reference herein.

### FIELD OF THE INVENTION

The invention relates generally to signaling proteins and antibodies, and their use to characterize and monitor disease.

### BACKGROUND OF THE INVENTION

Many cancers are characterized by disruptions in cellular signaling pathways that lead to aberrant control of cellular processes, or to uncontrolled growth and proliferation of cells. These disruptions are often caused by changes in the phosphorylation state, and thus the activity of, particular signaling proteins. Among these cancers are bone marrow cancers, such as chronic myelogenous leukemia (CML) and acute lymphocytic leukemia (ALL). There are about 4,700 new cases of CML in the United States annually, and it is estimated that 2,300 patients will die annually from the disease in the United States alone. See “Cancer Facts and Figures 2002,” American Cancer Society. There are about 3,500 new cases of ALL in the United States annually, and it is estimated that 1,400 patients will die annually from the disease in the United States alone. In children, leukemia is the most common type of cancer, and ALL is the most prevalent of these childhood leukemias. See id.

It has been directly demonstrated that the BCR-ABL oncoprotein, a protein tyrosine kinase, is the causative agent in human chronic myelogenous leukemia (CML). See Skorski et al., J. Clin Invest. 92:194-202 (1993); Snyder et al., Blood 82:600-605 (1993). The BCR-ABL oncoprotein is generated by the translocation of gene sequences from the cABL protein tyrosine kinase on chromosome 9 into BCR sequences on chromosome 22, producing the so-called Philadelphia chromsome. See, e.g. Kurzock et al., N. Engl. J. Med. 319: 990-998 (1988); Rosenberg et al., Adv. in Virus Res. 35: 39-81 (1988). The BCR-ABL oncogene has been found in at least 90-95% of cases of CML. See, e.g. Fialkow et al., Am. J. Med. 63:125-130 (1977). The translocation is also observed in approximately 20% of adults with acute lymphocytic leukemia (ALL), 5% of children with ALL, and 2% of adults with acute myelogenous leukemia (AML). See, e.g. Whang-Peng et al., Blood 36:448-457 (1970); Look, Semin. Oncol. 12: 92-104 (1985). The BCR-ABL gene produces two alternative chimeric proteins, P210 BCR-ABL, and P185 BCR-ABL, which are characteristic of CML and ALL, respectively. Tyrosine 245 of Abl is a major autophosphorylation site regulating activity of the kinase. See Brasher et al., J. Biol. Chem. 275(45): 35631-37(2000). Therefore, the Abl kinase is active when the tyrosine 245 site is phosphorylated (ibid).

BCR-ABL proteins exhibit heightened tyrosine kinase and transforming capabilities compared to the normal c-Abl protein. See, e.g. Konopka et al., Cell 37: 1035-1042 (1984). Many reports have indicated that BCR-ABL indeed acts as an oncogene and causes a variety of hematologic malignancies, including granulocytic hyperplasia resembling human CML, myelomonocytic leukemia, ALL, lymphomas, and erythroid leukemia, in vivo. See, e.g. Lugo et al., MCB 9:1263-1270 (1989); Daley et al., Science 247: 824-830 (1990); Honda, Blood 91: 2067-2075 (1998). As a result, BCR-ABL has become a target for the development of therapeutics to treat leukemia. Most recently, Gleevec® (ST1571), a small molecule inhibitor of the ABL kinase, has been approved for the treatment of CML. This drug is the first of a new class of targeted therapeutic agents designed to interfere with the pathways that mediate the growth, survival and metastases of tumor cells. The development of this drug represents a significant advance over the conventional therapies for CML and ALL including chemo-therapy and radiation, which are plagued by well-known side-effects and are often of limited effect since they fail to specifically target the underlying causes of the malignancies. However, Gleevec®, like many other therapeutics in development, targets a key signaling protein implicated in the progression of the disease.

The BCR-ABL tyrosine kinase itself is known to activate, via phosphorylation, various signaling molecules including the Ras/mitogen-activated protein kinase (MAPK) pathway, the phosphatidylinositol 3-kinase (Pl3-K)/Akt pathway, and signal transducers and activators of transcription (STATs, STAT1 and STAT5), as well acting as other oncogenes. See, e.g. Odajima et al., J. Biol. Chem. 275: 24096-105 (2000). Previous reports indicate that these downstream signaling proteins play an important role in BCR-ABL-mediated leukemogenesis. For example, it has been shown that a dominant negative (DN) form of Ras inhibited the growth and survival of BCR-ABL-transformed 32D cells. See, e.g. Cortez et al., Oncogene 13: 2589-2594 (1996). Similarly, DN STAT5 suppressed apoptosis resistance, factor-independent proliferation, and leukemogenic potential of a CML-derived cell line, K562, and BCR-ABL-transformed 32D and Ba/F3. See, e.g. de Groot et al., Blood 94: 1108-1112 (1999); Sillaber et al., Blood 95: 2118-2125 (2000). It has also been reported that a mutant form of BCR-ABL lacking the ability to activate Pl3-K failed to confer leukemogenic potentials on murine bone marrow cells in vitro and in vivo, indicating that Pl3-K/Akt pathway is also required for BCR-ABL-induced malignant transformation of hematopoietic cells. See, e.g. Skorski et al., EMBO J. 16: 6151-6161 (1997). Thus, multiple signaling pathway and protein activation disruptions appear to underlie these transformations.

It further appears that additional “adaptor proteins” interact directly with BCR-ABL and likely modulate its transforming activity. One such protein, CRKL, interacts directly with the BCR-ABL fusion protein and is constitutively phosphorylated in BCR-ABL-expressing cells. See, e.g. ten Hoeve et al., Oncogene 8: 2469-74 (1993). The correlation between CRKL phosphorylation and BCR-ABL expression in cancerous cells has been described, along with the diagnostic detection of phosphorylated CRKL alone using an antibody specific for phosphotyrosine itself. See U.S. Pat. No. 5,667,981, Groffen et al., Issued Sep. 16, 1997. CRKL also interacts with ABL. See id. Antibodies that bind non-phosphorylated CRKL have been described (see ten Hoeve, supra.). The phosphorylation of CRKL (as determined by mobility-shift Western blot) and the and the autophosphorylation of BCR-ABL (as determined by immunoprecipitation (IP) and p-Tyr Western blot) have recently been utilized as readouts of BCR-ABL inhibition by Gleevec®. See Sawyers, Science 293: 876-880 (2001). However, these approaches have several limitations, including the fact that shifts in electrophoretic mobility can be caused by post-translational modifications other than phosphorylation, hence the shift may not reflect actual activation of the kinase. Further, the assay is not very robust or sensitive: not all proteins show an electrophoretic mobility shift upon phosphorylation, and extensive phosphorylation can be necessary before a shift is observed. IP with a general phosphotyrosine antibody will detect any tyrosine phosphorylation, and thus may pull down the target protein phosphorylated at residues not relevant to activity. Thus, these approaches are not well suited to the clinical evaluation of BCR-ABL signaling. A polyclonal antibody to phospo-Stat5 (Tyr694) is commercially available (Santa Cruz Biotechnology, Inc., #sc-11761) as well as a monoclonal antibody (BD PharminGen #611964). However, use of these antibodies to determine BCR-ABL activity in clinical samples has not been adopted in the clinic.

Despite the increasing evidence that multiple signaling proteins and pathways mediate BCR-ABL-induced malignant transformation, the precise molecular events underlying these transformations have not been elucidated. As a result, therapeutics targeting only BCR-ABL may not be effective in treating malignancies involving other signaling proteins downstream of BCR-ABL. Indeed, recent clinical results have shown that patients may often develop resistance to Gleevec®. See, e.g. Sawyers, Science 294(5548):1834 (2001). The mechanism of resistance may vary from patient to patient, but is often a result of mutations in the BCR-ABL DNA that results in a variant kinase that is not affected by the inhibitor. See, e.g., Mercedes, Science 294(5548): 1834 (2001). Resistance may also occur through increased expression of the BCR-ABL protein. See, e.g. Keeshan, Leukemia (12):1823-33 (2001). Given the multiple signaling mechanisms that mediate signaling in CML, other mechanisms of resistance may not involve BCR-ABL kinase activity.

Accordingly, new reagents and assays are needed to elucidate the specific signaling pathway disruptions underlying CML and ALL and especially pathways mediating resistance to Gleevec® or other targeted therapeutics. Phospho-specific antibodies capable of detecting BCR-ABL signaling would allow the use of sensitive clinical techniques such as immunohistochemistry (IHC) and flow cytometry (FC), each of which have several advantages over mobility-shift assays. IHC enables the examination of expression of a particular phospho-protein in the context of the physiology of a tissue, providing immunostaining resolution down to the level of tissue structures, cell type, and even subcellular localization. The same level of resolution is not possible using homogenized cell/tissue extracts in mobility-shift or IP Western blots, thus important information about the pathogenesis of a disease and about signaling in the context of specific cell types or regions of the tissue known to play certain roles in disease may be missed. Similarly FC enables the detection of phosphoprotein markers in individual cells or cell types. FC using multiple antibodies with different fluorescent labels enables the examination of particular signaling pathways in specific cell types identified by well defined cell lineage markers. This powerful technique provides information on what proteins or phosphoproteins are co-expressed in the same cells. This information is not provided by Western blot staining with multiple antibodies, since the signals may come from distinct cell types that are combined in the homogenized tissue.

Antibodies and methods capable of detecting BCR-ABL, c-Abl, and Abl signaling pathway activation would also be useful in elucidating the mechanisms underlying patient resistance to BCR-ABL inhibitors, and selecting patients likely to respond to alternative combination therapies. Recent reports have described such combination therapies that either use improved BCR-ABL inhibitors or that target downstream pathways (Huron, DR et al. (2003) Cancer Res. in press and La Rosee, P et al. (2002) Clin Can Res 62, 7149-7153). The successful use of these combination therapies will require knowledge of the signaling events that are responsible for the Gleevec® resistance. Phospho-specific antibody-based assays and methods for identifying biomarkers of BCR-ABL, c-Abl, and Abl-mediated disease progression and responsiveness to therapeutics targeting the same would thus be highly desirable and would enable the selection of patient most likely to respond to therapeutics such as Gleevec®.

### SUMMARY OF THE INVENTION

The invention provides reagents and methods for detecting BCR-ABL or c-Abl kinase activity and/or Abl signaling pathway activation in a biological sample, such as a cell or tissue. The invention also provides methods for detecting the activity, or inhibition, of BCR-ABL or c-Abl kinase, and/or the Abl signaling pathway in a biological sample, and methods for predicting a patient likely to respond to a BCR-ABL inhibitor, or detection inhibition of the same, by using at least one antibody of the invention to determine the level of phosphorylated protein in the sample. Phospho-specific antibodies that bind to BCR-ABL and/or c-Abl, Bcr, CRKL, PYK2, Gab1, SHP2, Tyk2, MEK1/2 and ERK1/2 when these proteins are phosphorylated at specific tyrosines, serines or threonines (Table 1) are provided. These proteins have now been identified as biomarkers of c-Abl pathway-mediated disease progression and BCR-ABL inhibitor/therapeutic responsiveness. Biological samples may be taken from a subject having, or at risk of cancer, for example CML or ALL. An exemplary inhibitor is an ABL kinase inhibitor, such as Gleevec® (STI-571). Kits for carrying out the methods of the invention are also provided.

### BRIEF DESCRIPTION OF THE DRAWINGS

FIG. 1—depicts a Western blot analysis of proteins whose phosphorylation is affected by Gleevec®. Membranes probed with antibodies to total CRKL or phosphorylated CRKL. Size of detected protein matches predicted molecular weight (39 kDa) as determined by comparison to marker (m=40 kDa).

FIG. 2—depicts an immunohistochemical (IHC) analysis of Abl signaling pathway activation in: (A) paraffin-embedded K562 cells untreated or treated with the Abl inhibitor Gleevec® using phosphorylation-specific antibodies of the invention, and (B) paraffin-embedded human bone marrow tissue from a CML patient stained with a phospho-specific antibody to Bcr (Tyr177).

FIG. 3—depicts an immunohistochemical analysis of cell smears of patient lymphocytes probed with phospho-specific antibodies to c-Abl and CRKL.

FIG. 4—depicts a flow cytometric analysis of K562 cells untreated or treated with Gleevec®, using phospho-CRKL(Tyr207), phospho-Bcr (Y177) and phospho-c-Abl(Tyr245) antibodies of the invention.

FIG. 5—depicts a flow analysis of CML patient samples with antibodies against phospho-abl (Tyr245), phospho-Bcr (Y177) and phospho-CRKL (Tyr207). Figure A shows histograms from patients #6, #9, #11. Figure B is a table summarizing the cytometric analysis.

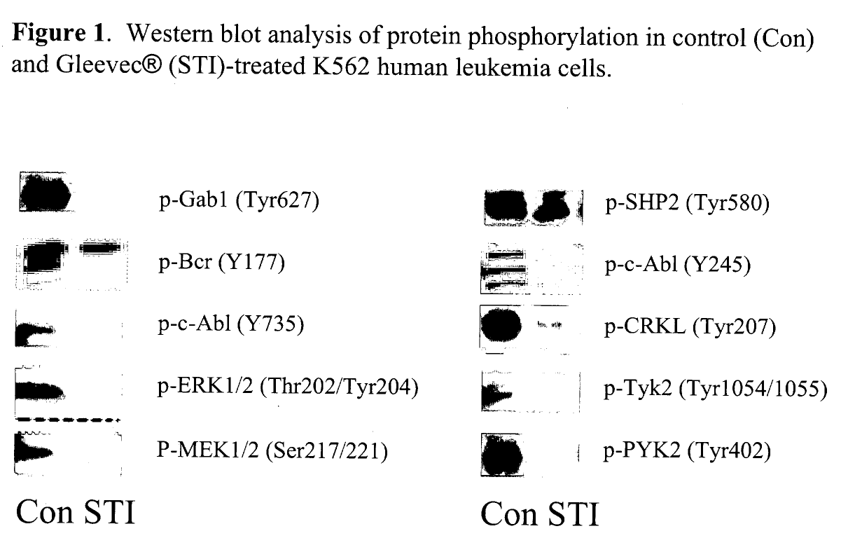

### DETAILED DESCRIPTION OF THE INVENTION

As disclosed herein, it has now been discovered that c-Abl, Bcr, Gab1, PYK2, TYK2, SHP2, ERK1/2 and/or MEK1/2, when phosphorylated at certain tyrosine, serines, and/or threonine residues, are useful biomarkers of c-Abl signaling pathway-mediated disease progression, and may be also be exploited as predictors of patient response to a therapeutic having activity against a disease involving altered Abl pathway signaling. BCR-ABL and CRKL, when phosphorylated at certain residues, may also be employed. Accordingly, the invention provides, in part, phospho-specific antibodies that bind BCR-ABL or proteins downstream of BCR-ABL when phosphorylated at specific residues, namely: Bcr (Tyr 177), PYK2 (Tyr402), Tyk2 (Tyr1054/1055); SHP2 (Tyr580), Gab1 (Tyr627), ERK1/2 (Thr 202/Tyr204), MEK1/2 (Ser217/221), BCR-ABL (Tyr245) and/or c-Abl (Tyr245), BCR-ABL (Thr735) and/or c-Abl (Thr735), and CRKL (Tyr207).

Tyrosine 245 of Abl is a major autophosphorylation site regulating Abl kinase activity, and is present in both c-Abl and BCR-ABL. Threonine 735 of c-Abl is a 14-3-3 binding site that is thought to regulate Abl localization and activity. See Brasher et al., supra. Activated Abl, as with BCR-ABL, phosphorylates CRKL on tyrosine 207. See dejong et al., Oncogene 14(5): 507-13 (1975). Bcr phosphorylation at tyrosine 177 occurs in the BCR-ABL translocation and regulates Gab1 and GRB2 binding (He, Y. et al. (2002) Blood 99, 2957-2968 and Sattler, M. et al. (2002) Cancer Cell 1, 479-492).

Gab1 is an adaptor protein that binds PLC gamma, Pl-3-kinase and SHP2 when phosphorylated at Tyr307, Tyr472 and Tyr627 respectively (see Ingham, R. J. et al. (2001) J. Biol. Chem. 276, 12257-12265 and Lehr, S. et al. (1999) Biochemistry 38, 151-159). A potential role for Gab1 in BCR-ABL transformation has been suggested by a mass spectrometry profile of tyrosine phosphoryalation in 32D cells (see Salomon, A R et al. (2003) PNAS 100, 443-448). A potential role for cytokine signaling and TYK2 in oncogenesis has been suggested by expression studies in the II-3 dependent cell line, Ba/F3 cells (Lacronique, V et al. (2000) Blood 95 (6), 2076-83). In this system expression of the TYK2 kinase domain fused to the TEL oligomerization domain was sufficient to substitute for II-3. The role of the MAP kinase pathway (ERK1/2 and MEK1/2) in BCR-ABL transformation has been well documented (Woessmann, W and N F Mivechi (2001) Exp Cell Res 264 (2),193-200).

The use of MEK inhibitors in combination with Gleevec® for the treatment of BCR-ABL positive leukemia cells has been successfully tested on cell lines such as K562 cells (Yu, C (2002) Cancer Biol Ther 1(6), 674-682. The activation of Gad1 and it's binding to SHP2 has been shown to be required for the activation of the MAP kinase pathway (Maroun, C. R. et al. (2000) Mol. Cell. Biol. 20, 8513-8525). Therefore, the combination of proteins detected by the phospho-specific antibodies of the invention provide a broad characterization of the signaling activity that mediates BCR-ABL activity and cellular transformation.

The phospho-specific antibodies of the invention enable the study of BCR-ABL and c-Abl kinase activity and Abl signaling pathway activation in cells and tissue using the highly sensitive techniques of IHC and flow cytometry. Thus, the invention also provides methods for detecting and profiling the activity of these signaling proteins and pathways in cells or tissues, e.g. from CML or ALL patients, including inhibitor responsive or resistant patients, as further described below. The methods and kits of the invention employ one or more phospho-specific antibodies to detect the level of one or more of phosphorylated c-Abl, Bcr, Gab1, PYK2, TYK2, SHP2, ERK1/2 and/or MEK1/2, and optionally, at least one of BCR-ABL or CRKL, in order to identify relevant biomarkers of disease progression or therapeutic responsiveness, or to predict a patient a likely to respond to a BCR-ABL or c-Abl kinase inhibitor, such as Gleevec®.

These reagents and methods are an important advance over current methods of detecting Abl signaling via mobility-shift Western blot or immunoprecipitation Western blot, which are not sufficiently sensitive, impractical for clinical practice and not informative enough to be medically useful. Indeed, historical experience with Gleevec® has indicated that patient resistance to the drug in various patient groups is a result of not one, but likely several unelucidated signaling proteins downstream of BCR-ABL and c-Abl. The methods, kits, and reagents provided by the invention will be highly useful, inter alia, in powerful new approaches to identifying and selecting patients most likely to respond to therapeutics targeting c-Abl signaling.

The further aspect, advantages, and embodiments of the present invention are described in more detail below. All references cited above and below are hereby incorporated herein by reference.

A. Antibodies and Cell Lines

The invention provides, in part, the following phospho-specific antibodies as listed in Table 1:

(i) an antibody that binds BCR-ABL and/or c-Abl when phosphorylated at tyrosine 245 (Tyr245), but does not bind BCR-ABL or c-Abl when not phosphorylated at this position, nor to BCR-ABL or c-Abl phosphorylated at other residues (hereinafter referred to as a “BCR-ABL (Tyr245) or c-Abl (Tyr245) phospho-specific antibody”);

(ii) an antibody that binds BCR-ABL and/or c-Abl when phosphorylated at threonine 735 (Thr735), but does not bind BCR-ABL or c-Abl when not phosphorylated at this position, nor to BCR-ABL or c-Abl phosphorylated at other residues (hereinafter referred to as a “BCR-ABL (Thr735) or c-Abl (Thr735) phospho-specific antibody”);

(iii) an antibody that binds Bcr when phosphorylated at tyrosine 177 (Tyr177), but does not bind Bcr when not phosphorylated at this position, nor to Bcr phosphorylated at other residues (hereinafter referred to as a “Bcr (Tyr177) phospho-specific antibody”);

(iv) an antibody that binds CRKL when phosphorylated at tyrosine 207 (Tyr207), but does not bind CRKL when not phosphorylated at this position, nor to CRKL phosphorylated at other residues (hereinafter referred to as a “CRKL (Tyr207) phospho-specific antibody”);

(v) an antibody that binds PYK2 when phosphorylated at tyrosine 402 (Tyr402), but does not bind PYK2 when not phosphorylated at this position, nor to PYK2 phosphorylated at other residues (hereinafter referred to as a “PYK2 (Tyr402) phospho-specific antibody”);

(vi) an antibody that binds Tyk2 when phosphorylated at tyrosine 1054/1055 (Tyr1054/1055), but does not bind Tyk2 when not phosphorylated at this position, nor to Tyk2 phosphorylated at other residues (hereinafter referred to as a “Tyk2 (Tyr1054/1055) phospho-specific antibody”);

(vii) an antibody that binds SHP2 when phosphorylated at tyrosine 580 (Tyr580), but does not bind SHP2 when not phosphorylated at this position, nor to SHP2 phosphorylated at other residues (hereinafter referred to as a “SHP2 (Tyr580) phospho-specific antibody”);

(viii) an antibody that binds Gab1 when phosphorylated at tyrosine 627 (Tyr627), but does not bind Gab1 when not phosphorylated at this position, nor to Gab1 phosphorylated at other residues (hereinafter referred to as a “Gab1 (Tyr627) phospho-specific antibody”);

(ix) an antibody that binds ERK1/2 when phosphorylated at threonine 202 and tyrosine 204 (Thr 202/Tyr204), but does not bind ERK1/2 when not phosphorylated at this position, nor to ERK1/2 phosphorylated at other residues (hereinafter referred to as an “ERK1/2 (Tyr202/Tyr204) phospho-specific antibody”); and

(x) an antibody that binds MEK1/2 when phosphorylated at serine 217 and 221 (Ser217/221), but does not bind MEK1/2 when not phosphorylated at this position, nor to MEK1/2 phosphorylated at other residues (hereinafter referred to as a “MEK1/2 (Ser217/221) phospho-specific antibody”).

The above-identified antibodies may be monoclonal or polyclonal. The term “antibody” or “antibodies” as used herein refers to all types of immunoglobulins, including IgG, IgM, IgA, IgD, and IgE. The antibody may be may be of any species of origin, including (for example) mouse, rat, rabbit, horse, or human, or may be chimeric antibodies. See, e.g., M. Walker et al., Molec. Immunol. 26: 403-11 (1989); Morrision et al., Proc. Nat'l. Acad. Sci. 81: 6851 (1984); Neuberger et al., Nature 312:604 (1984)). The antibody may be a recombinant monoclonal antibody produced according to the methods disclosed in U.S. Pat. No. 4,474,893 (Reading) or U.S. Pat. No. 4,816,567 (Cabilly et al.) The antibodies may also be chemically constructed by specific antibodies made according to the method disclosed in U.S. Pat. No. 4,676,980 (Segel et al.).

The term includes fragments of the antibody which bind to the antigen (or more preferably the epitope) bound by particular antibodies disclosed herein. Such antibodies and antibody fragments may be produced by a variety of techniques well known in the art, as discussed below. Antibody fragments that bind to the phosphorylated epitope (i.e., the specific binding site) bound by an antibody disclosed herein can be identified in accordance with known techniques, such as their ability to compete with labeled monoclonal antibody in a competitive binding assay.

As used herein, the phrase “antibody (or antibodies) of the invention” refers collectively to the phospho-specific antibodies listed in Table 1 meaning the respective phospho-specific antibodies described therein, and are used interchangeably with the same. The term “does not bind” with respect to such an antibody means does not substantially react with as compared to the binding of such an antibody to the target when phosphorylated at the appropriate residue.

The preferred epitopic site of the c-Abl (Tyr245) antibodies of the invention is a peptide fragment consisting essentially of about 11 to 17 amino acids including the phosphorylated tyrosine 245, wherein about 5 to 8 amino acids are positioned on each side of the tyrosine phosphorylation site (c-Abl protein sequence published at SwissProt #P00519). The preferred epitopic site of the c-Abl (Thr735) antibodies of the invention is a peptide fragment consisting essentially of about 11 to 17 amino acids including the phosphorylated threonine 735, wherein about 5 to 8 amino acids are positioned on each side of the tyrosine phosphorylation site (c-Abl protein sequence published at SwissProt #P00519). The preferred epitopic site of the Bcr (Tyr177) antibodies of the invention is a peptide fragment consisting essentially of about 11 to 17 amino acids including the phosphorylated tyrosine 177, wherein about 5 to 8 amino acids are positioned on each side of the tyrosine phosphorylation site (Bcr protein sequence published at SwissProt #).

The preferred epitopic site of the CRKL (Tyr207) antibodies of the invention is a peptide fragment consisting essentially of about 11 to 17 amino acids including the phosphorylated tyrosine 207, wherein about 5 to 8 amino acids are positioned on each side of the tyrosine phosphorylation site (CRKL protein sequence published at SwissProt #P46109). The preferred epitopic site of the Gab1 antibodies of the invention is a peptide fragment consisting essentially of about 11 to 17 amino acids including the phosphorylated tyrosine 402, wherein about 5 to 8 amino acids are positioned on each side of the tyrosine phosphorylation site (Gab1 protein sequence published at SwissProt #Q13480). The preferred epitopic site of the SHP2 (Tyr580) antibodies of the invention is a peptide fragment consisting essentially of about 11 to 17 amino acids including the phosphorylated tyrosine 580, wherein about 5 to 8 amino acids are positioned on each side of the tyrosine phosphorylation site (SHP2 protein sequence published at SwissProt #Q06124).

The preferred epitopic site of the Tyk2 (Tyr1054/1055) antibodies of the invention is a peptide fragment consisting essentially of about 11 to 17 amino acids including the phosphorylated tyrosine 245, wherein about 5 to 8 amino acids are positioned on each side of the tyrosine phosphorylation site (Tyk2 protein sequence published at SwissProt #P29597). The preferred epitopic site of the PYK2 (Tyr402) antibodies of the invention is a peptide fragment consisting essentially of about 11 to 17 amino acids including the phosphorylated tyrosine 402, wherein about 5 to 8 amino acids are positioned on each side of the tyrosine phosphorylation site (PYK2 protein sequence published at SwissProt #Q14289).

The preferred epitopic site of the ERK1/2 (Thr202/Tyr204) antibodies of the invention is a peptide fragment consisting essentially of about 11 to 17 amino acids including the phosphorylated threonine 202 and tyrosine 204, wherein about 5 to 8 amino acids are positioned on each side of the phosphorylation sites (ERK1 protein sequence published at SwissProt #P27361). The preferred epitopic site of the MEK1/2 (Ser217/221) antibodies of the invention is a peptide fragment consisting essentially of about 11 to 17 amino acids including the phosphorylated serine 217 and 221, wherein about 5 to 8 amino acids are positioned on each side of the serine phosphorylation sites (MEK1 protein sequence published at SwissProt #Q02750).

Monoclonal antibodies of the invention may be produced in a hybridoma cell line according to the well-known technique of Kohler and Milstein. Nature 265:495-97 (1975); Kohler and Milstein, Eur. J. Immunol. 6: 511 (1976); see also, CURRENT PROTOCOLS IN MOLECULAR BIOLOGY, Ausubel et al. Eds. (1989). Monoclonal antibodies so produced are highly specific, and improve the selectivity and specificity of diagnostic assay methods provided by the invention. For example, a solution containing the appropriate antigen may be injected into a mouse and, after a sufficient time (in keeping with conventional techniques), the mouse sacrificed and spleen cells obtained. The spleen cells are then immortalized by fusing them with myeloma cells, typically in the presence of polyethylene glycol, to produce hybridoma cells. The hybridoma cells are then grown in a suitable selection media, such as hypoxanthine-aminopterin-thymidine (HAT), and the supernatant screened for monoclonal antibodies having the desired specificity, as described below. The secreted antibody may be recovered from tissue culture supernatant by conventional methods such as precipitation, ion exchange or affinity chromatography, or the like.

Monoclonal Fab fragments may also be produced in Escherichia coli by recombinant techniques known to those skilled in the art. See, e.g., W. Huse, Science 246:1275-81 (1989); Mullinax et al., Proc. Nat'l Acad. Sci. 87: 8095 (1990). If monoclonal antibodies of one isotype are preferred for a particular application, particular isotypes can be prepared directly, by selecting from the initial fusion, or prepared secondarily, from a parental hybridoma secreting a monoclonal antibody of different isotype by using the sib selection technique to isolate class-switch variants (Steplewski, et al., Proc. Nat'l. Acad. Sci., 82: 8653 (1985); Spira et al., J. Immunol. Methods, 74: 307 (1984)).

Polyclonal antibodies of the invention may be produced according to standard techniques by immunizing a suitable animal (e.g., rabbit, goat, etc.) with an antigen encompassing the phospho-epitope as listed in Table 1, collecting immune serum from the animal, and separating the polyclonal antibodies from the immune serum, in accordance with known procedures. In a preferred embodiment, the antigen is a phospho-peptide antigen comprising the phosphorylation site and surrounding sequence, the antigen being selected and constructed in accordance with well known techniques. See, e.g., ANTIBODIES: A LABORATORY MANUAL, Chapter 5, p. 75-76, Harlow & Lane Eds., Cold Spring Harbor Laboratory (1988); Czernik, Methods In Enzymology, 201: 264-283 (1991); Merrifield, J. Am. Chem. Soc. 85: 21-49 (1962)). Particularly preferred peptide antigens for each protein are described in the Examples, below. It will be appreciated by those of skill in the art that longer or shorter phosphopeptide antigens may be employed. See Id. A polyclonal antiserum produced as described herein may be screened as further described below.

Antibodies of the invention may be screened for epitope and phospho-specificity according to standard techniques. See, e.g. Czernik et al., Methods in Enzymology, 201: 264-283 (1991). For example, the antibodies may be screened against the phospho and non-phospho peptide library by ELISA to ensure specificity for both the desired antigen (i.e. that epitope including Tyr245 in the case of c-Abl, for example) and for reactivity only with the phosphorylated form of the antigen. Peptide competition assays may be carried out to confirm lack of reactivity with other phospho epitopes on the respective proteins.

The antibodies may also be tested by Western blotting against cell preparations containing the phosphorylated target protein, e.g. cell lines expressing these proteins, to confirm reactivity with the desired phosphorylated target. Specificity against the desired phosphorylated epitopes may also be examined by construction of mutants lacking phosphorylatable residues at positions outside the desired epitope known to be phosphorylated, or by mutating the desired phospho-epitope and confirming lack of reactivity. Cross-reactivity with proteins other than the specified target proteins, is readily characterized by Western blotting alongside markers of known molecular weight. Amino acid sequences of cross-reacting proteins may be examined to identify sites highly homologous to the sequences surrounding the specific phosphorylation sites on the respective protein.

Antibodies of the invention may be further characterized via immunohistochemical (IHC) staining using normal and diseased tissues to examine phosphorylation and activation status of these key signaling molecules in diseased tissue. IHC may be carried out according to well known techniques. See, e.g., ANTIBODIES: A LABORATORY MANUAL, Chapter 10, Harlow & Lane Eds., Cold Spring Harbor Laboratory (1988). Briefly, paraffin-embedded tissue (e.g. tumor tissue) is prepared for immunohistochemical staining by deparaffinizing tissue sections with xylene followed by ethanol; hydrating in water then PBS; unmasking antigen by heating slide in sodium citrate buffer or EDTA; incubating sections in hydrogen peroxide; blocking in blocking solution; incubating slide in primary antibody and secondary antibody; and finally detecting using ABC avidin/biotin method according to manufacturer's instructions. Alternatively, the antibodies may be characterized on cell smears according to standard clinical practices (ibid). In this procedure, fresh or fixed cells are spotted (smeared) onto glass slides, allowed to air dry and fixed, blocked and stained on the slide. The slides are then processed as described above.

Additional, non-phospho-specific antibodies or reagents may also be utilized in the methods of the present invention. For example, other modification-specific antibodies may be included, such as acetylation- or nitrosylation-specific antibodies, to detect activation of signal transduction targets having such modifications. Control antibodies may also be included, for example, protein-specific antibodies that detect merely the presence of a given signal transduction protein (not its modification status), or site-specific antibodies that detect a target in its unphosphorylated form. The detection of particular biomarkers (as well as additional proteins) may be done sequentially, simultaneously, or certain subsets may be done in tandem.

B. Detection & Profiling Methods

The methods disclosed herein may be employed with any biological sample (preferably a cell or tissue or lysate of the same) suspected of containing phosphorylated BCR-ABL, c-Abl, Bcr, CRKL, Gab1, PYK2, TYK2, SHP2, ERK1/2 and/or MEK1/2. Cells may be obtained from human subjects for use in the methods disclosed herein from a variety of sources, for example, from blood, fine needle aspirate, ductal lavage, bone marrow sample, or ascites fluid, etc. In the alternative, the sample taken from the subject can be a tissue sample (e.g., a biopsy tissue), such as skin or hair follicle or tumor tissue. As used herein “cell or tissue” means any biological sample comprising one or more cells, including lysates of the same.

In accordance with the present invention, certain novel biomarkers relevant to c-Abl mediated signaling and disease progression have now been identified. These downstream markers are more powerfully informative than single markers, such as BCR-ABL or CRKL, which have proven to be of limited value in prognosis or prediction of patient response. The present findings evidence that detection of one or more relevant downstream markers is more information of signaling events in this pathway relevant to disease and therapeutic response, and hence to prediction of the same in patients. This finding is consistent with historical observations indicating that Gleevec® effectiveness and resistance in patients is being mediated by multiple signal transduction proteins, previously unidentified.

Accordingly, in one embodiment, the invention provides a method for detecting the activity of BCR-ABL or c-Abl kinase and/or the Abl signaling pathway in a cell or tissue, the method comprising the steps of:

(a) obtaining at least one test cell or tissue from a subject;

(b) contacting said test cell or tissue with at least one phospho-specific antibody selected from the group consisting of:

(i) a Bcr (Tyr177) phospho-specific antibody;

(ii) a PYK2 (Tyr402) phospho-specific antibody;

(iii) a Tyk2 (Tyr1054/1055) phospho-specific antibody;

(iv) a SHP2 (Tyr580) phospho-specific antibody;

(v) a Gab1 (Tyr627) phospho-specific antibody;

(vi) an ERK1/2 (Thr 202/Tyr204) phospho-specific antibody;

(vii) a MEK1/2 (Ser217/221) phospho-specific antibody;

and, optionally, at least one phospho-specific antibody selected from the group consisting of:

(viii) a BCR-ABL (Tyr245) and/or c-Abl (Tyr245) phospho-specific antibody;

(ix) a BCR-ABL (Thr735) and/or c-Abl (Thr735) phospho-specific antibody;

(x) a CRKL (Tyr207) phospho-specific antibody;

(c) determining the level of at least one of phosphorylated c-Abl, Bcr, Gab1, PYK2, TYK2, SHP2, ERK1/2 and/or MEK1/2, and optionally, at least one of BCR-ABL or CRKL, bound by the antibody of step (b); and

(d) comparing the level of phosphorylated protein determined in step(c) for said test cell or tissue with the level of phosphorylated protein in a reference sample, thereby detecting the activity of BCR-ABL, c-Abl, and/or the c-Abl signaling pathway in said test cell or tissue.

In certain preferred embodiments, two or more of phosphorylated c-Abl, Bcr, Gab1, PYK2, TYK2, SHP2, ERK1/2 and/or MEK1/2 are detected in step (b). In other preferred embodiments, phospho-antibodies against three or more of these proteins are employed. In still other preferred embodiments, phosphorylation of all of c-Abl, Bcr, Gab1, PYK2, TYK2, SHP2, ERK1/2 and/or MEK1/2 are detected in step (b). BCR-ABL and/or CRKL phosphorylation may optionally be examined, since neither of these markers alone is highly informative, but either or both may provide useful information when taken together with other downstream signaling events. The particular number or subsets of target biomarkers to be examined will depend, in part, on the cell or tissue being examined (or the disease or treatment). Although all of the markers may be examined to provide the most informative c-Abl signaling profile, particular subsets may be employed as those subsets are identified as the best collective biomarkers for a given disease, therapeutic, or subset of patients. It is anticipated such subsets will be identified as future work in c-Abl signaling using the methods of the present invention continue.

In a preferred embodiment, the subject has, or is at risk of, cancer. In another preferred embodiment, the cancer is chronic myelogenous leukemia (CML) or acute lymphocytic leukemia (ALL). In still another preferred embodiment, the determination of phosphorylated protein levels in step (b) comprises conducting immunohistochemistry (IHC) and/or flow cytometry.

In another embodiment, the invention provides a method for detecting the inhibition of BCR-ABL or c-Abl kinase by an inhibitor, said method comprising the steps of: (a) obtaining at least one test cell or tissue from a subject; (b) contacting said test cell or tissue with said inhibitor and at least one phospho-specific antibody selected from the group consisting of:

(i) a Bcr (Tyr177) phospho-specific antibody;

(ii) a PYK2 (Tyr402) phospho-specific antibody;

(iii) a Tyk2 (Tyr1054/1055) phospho-specific antibody;

(iv) a SHP2 (Tyr580) phospho-specific antibody;

(v) a Gab1 (Tyr627) phospho-specific antibody;

(vi) an ERK1/2 (Thr 202/Tyr204) phospho-specific antibody;

(vii) a MEK1/2 (Ser217/221) phospho-specific antibody;

and, optionally, at least one phospho-specific antibody selected from the group consisting of:

(viii) a BCR-ABL (Tyr245) and/or c-Abl (Tyr245) phospho-specific antibody;

(ix) a BCR-ABL (Thr735) and/or c-Abl (Thr735) phospho-specific antibody;

(x) a CRKL (Tyr207) phospho-specific antibody;

(c) conducting a cellular assay with said test cell or tissue to determine the level of at least one of phosphorylated c-Abl, Bcr, Gab1, PYK2, Tyk2, SHP2, ERK1/2 and/or MEK1/2, and optionally at least one of BCR-ABL or CRKL, bound by the antibody of step (b); and

(d) comparing the level of phosphorylated protein determined in step(c) for said test cell or tissue with the level of phosphorylated protein in a reference sample not treated with said inhibitor, thereby detecting the inhibition of BCR-ABL or c-Abl kinase by said inhibitor in said test cell or tissue.

In yet another embodiment, the invention provides a method for identifying a patient likely to respond to a BCR-ABL kinase inhibitor for the treatment of CML or ALL, said method comprising the steps of: (a) obtaining at least one test cell or tissue from a patient having CML or ALL; (b) contacting said test cell or tissue with at least one phospho-specific antibody selected from the group consisting of:

(i) a Bcr (Tyr177) phospho-specific antibody;

(ii) a PYK2 (Tyr402) phospho-specific antibody;

(iii) a Tyk2 (Tyr1054/1055) phospho-specific antibody;

(iv) a SHP2 (Tyr580) phospho-specific antibody;

(v) a Gab1 (Tyr627) phospho-specific antibody;

(vi) an ERK1/2 (Thr 202/Tyr204) phospho-specific antibody;

(vii) a MEK1/2 (Ser217/221) phospho-specific antibody;

and, optionally, at least one phospho-specific antibody selected from the group consisting of:

(viii) a BCR-ABL (Tyr245) and/or c-Abl (Tyr245) phospho-specific antibody;

(ix) a BCR-ABL (Thr735) and/or c-Abl (Thr735) phospho-specific antibody;

(x) a CRKL (Tyr207) phospho-specific antibody;

(c) conducting a cellular assay with said test cell or tissue to determine the level of at least one of phosphorylated c-Abl, Bcr, Gab1, PYK2, Tyk2, SHP2, ERK1/2 and/or MEK1/2, and optionally at least one of phosphorylaetd BCR-ABL or CRKL, bound by the antibody of step (b), wherein a significantly high level of one or more of these phosphorylated proteins identifies a patient likely to respond to a BCR-ABL kinase inhibitor for the treatment of CML or ALL.

In one preferred embodiment, the aforementioned method further comprises the step of (d) comparing the level of phosphorylated protein determined in step(c) for said test cell or tissue with the level of phosphorylated protein in a reference sample characteristic of CML or ALL patients responsive to a BCR-ABL inhibitor. Suitable cellular assays are described in “Immunoassay Formats” below. In certain preferred embodiments, the cellular assays comprises immunohistochemistry (IHC) or flow cytometry (FC).

In other preferred embodiments of the foregoing predictive or inhibition detection methods, step (b) comprises contacting said test cell or tissue with three or more phospho-specific antibodies comprising said Bcr (Tyr177) phospho-specific antibody, said c-Abl (Tyr245) and/or (Thr735) phospho-specific antibody, and said CRKL (Tyr207) phospho-specific antibody. This collection of biomarkers was identified as relevant to CML patient resistance to Gleevec® (see Example 3).

As noted above, the predictive methods of the invention may employ phospho-specific antibodies to either a single biomarker, or any combination of biomarkers identified in (i)-(vii) above, depending on the particular patient for which prediction of response is desired, or upon the particular inhibitor at issue. It is anticipated that certain subsets or combinations of the biomarkers disclosed herein may subsequently be identified as the best and most information biomarkers for a given disease, therapeutic, or patient subset. Such specific combinations and subsets are within the scope of the present invention.

The invention also provides a method for identifying a protein biomarker of patient response or resistance to a BCR-ABL inhibitor for the treatment of CML or ALL, said method comprising the steps of:

(a) obtaining at least one test cell or tissue from (i) each of a plurality of BCR-ABL inhibitor-responsive patients having CML or ALL, (ii) each of a plurality of BCR-ABL inhibitor-resistant patients having CML or ALL, and (iii) control patients having neither disease;

(b) contacting said test cells or tissues with two or more phospho-specific antibodies selected from group consisting of:

(i) a Bcr (Tyr177) phospho-specific antibody;

(ii) a PYK2 (Tyr402) phospho-specific antibody;

(iii) a Tyk2 (Tyr1054/1055) phospho-specific antibody;

(iv) a SHP2 (Tyr580) phospho-specific antibody;

(v) a Gab1 (Tyr627) phospho-specific antibody;

(vi) an ERK1/2 (Thr 202/Tyr204) phospho-specific antibody;

(vii) a MEK1/2 (Ser217/221) phospho-specific antibody;

and, optionally, at least one phospho-specific antibody selected from the group consisting of:

(viii) a BCR-ABL (Tyr245) and/or c-Abl (Tyr245) phospho-specific antibody;

(ix) a BCR-ABL (Thr735) and/or c-Abl (Thr735) phospho-specific antibody;

(x) a CRKL (Tyr207) phospho-specific antibody;

(c) conducting a cellular assay with said test cells or tissues to determine the level of two or more of phosphorylated c-Abl, Bcr, Gab1, PYK2, Tyk2, SHP2, ERK1/2 and/or MEK1/2, and optionally at least one of phosphorylated BCR-ABL or CRKL, bound by the antibodies of step (b), thereby generating an activation profile for said inhibitor-responsive and inhibitor-resistant patients and said control patients; and

(d) comparing said activation profiles of step (c), whereby a substantial difference in the activation profiles for said inhibitor-responsive and said inhibitor-resistant patients as compared to said control patients identifies one or more signal transduction protein(s) as being associated with patient responsiveness or resistance to a BCR-ABL inhibitor for the treatment of CML or ALL.

In certain preferred embodiments of the above methods, the inhibitor comprises an ABL kinase inhibitor. In other preferred embodiments, the kinase inhibitor is Gleevec® (STI-571). As used throughout this specification, the terms “inhibitor” and “therapeutic” mean any composition of one or more compounds, inhibitors or therapeutics, including cocktail therapies (which may also include one or more chemotherapeutic agents). Certain of the disclosed biomarkers, or subsets of the same, may prove to be the best predictors of patient response or resistance to a given therapeutic for a given disease. Others may prove to be the best biomarkers for a different therapeutic or disease. Such subsets and collections of the disclosed biomarkers are within the scope of the present invention.

In certain preferred embodiments of the methods of the invention, the test cell or tissue is a cell or tissue suspected of having altered BCR-ABL phosphorylation, such as bone marrow from a CML or ALL patient.

The methods described above are applicable to examining tissues or samples from BCR-ABL related cancers, such as leukemias, in which activity of BCR-ABL, c-Abl, or the c-Abl signaling pathway has predictive value as to the response of the disease to therapy. It is anticipated that the antibodies of the invention will have diagnostic utility in a disease characterized by, or involving, altered BCR-ABL activity or altered BCR-ABL, c-Abl, Bcr, CRKL, Gab1, PYK2, TYK2, SHP2, ERK1/2 and/or MEK1/2 phosphorylation.

The methods are applicable, for example, where samples are taken from a subject has not been previously diagnosed as having leukemia, nor has yet undergone treatment for leukemia, and the method is employed to help diagnose the disease, monitor the response of the patient to BCR-ABL targeted therapy, or assess risk of the subject developing such resistance to the targeted. Such diagnostic assay may be carried out prior to preliminary blood, skin biopsy evaluation or surgical surveillance procedures. Such a diagnostic assay may be employed to identify patients with activated BCR-ABL who would be most likely to respond to cancer therapeutics targeted at inhibiting BCR-ABL activity. Such a selection of patients would be useful in the clinical evaluation of efficacy of future BCR-ABL inhibitors as well as in the future prescription of such drugs to patients. Alternatively, the methods are applicable where a subject has been previously diagnosed as having leukemia, and possibly has already undergone treatment for the disease, and the method is employed to monitor the progression of such cancer involving BCR-ABL, or the treatment thereof.

The invention further provides, in another embodiment, a method for identifying a compound which modulates BCR-ABL or c-Abl activity in a cell or tissue by (a) contacting the test cell or tissue with the compound, (b) detecting the level of at least one biomarker disclosed herein in said test tissue of step (a) using at least one phospho-specific antibody of the invention under conditions suitable for formation of an antibody-protein complexes, and (c) comparing the level of phosphorylated proteins detected in step (b) with the presence of phosphorylated proteins in a control tissue not contacted with the compound, wherein a difference in c-Abl, Gab1, PYK2, TYK2, SHP2, ERK1/2 and/or MEK1/2, and optionally BCR-ABL and/or CRKL, phosphorylation levels between the test and control tissues identifies the compound as a modulator of BCR-ABL or c-Abl activity. Conditions suitable for the formation of antibody-antigen complexes are well known in the art (see part (d) below and references cited therein).

C. Immunoassay Formats & Diagnostic Kits

Assays carried out in accordance with methods of the present invention may be homogeneous assays or heterogeneous assays. In a homogeneous assay the immunological reaction usually involves a phospho-specific antibody of the invention, a labeled analyte, and the sample of interest. The signal arising from the label is modified, directly or indirectly, upon the binding of the antibodies to the labeled analyte. Both the immunological reaction and detection of the extent thereof are carried out in a homogeneous solution. Immunochemical labels that may be employed include free radicals, radioisotopes, fluorescent dyes, enzymes, bacteriophages, coenzymes, and so forth.

In a heterogeneous assay approach, the reagents are usually the specimen, the antibodies of the invention, and suitable means for producing a detectable signal. Similar specimens as described above may be used. The antibody is generally immobilized on a support, such as a bead, plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the specimen. Means for producing a detectable signal include the use of radioactive labels, fluorescent labels, enzyme labels, and so forth. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are the radioimmunoassay, immunofluorescence methods, enzyme-linked immunoassays, and the like.

Immunoassay formats and variations thereof which may be useful for carrying out the methods disclosed herein are well known in the art. See generally E. Maggio, Enzyme-Immunoassay, (1980) (CRC Press, Inc., Boca Raton, Fla.); see also, e.g., U.S. Pat. No. 4,727,022 (Skold et al., “Methods for Modulating Ligand-Receptor Interactions and their Application”); U.S. Pat. No. 4,659,678 (Forrest et al., “Immunoassay of Antigens”); U.S. Pat. No. 4,376,110 (David et al., “Immunometric Assays Using Monoclonal Antibodies”). Conditions suitable for the formation of reagent-antibody complexes are well described. See id. Monoclonal antibodies of the invention may be used in a “two-site” or “sandwich” assay, with a single cell line serving as a source for both the labeled monoclonal antibody and the bound monoclonal antibody. Such assays are described in U.S. Pat. No. 4,376,110. The concentration of detectable reagent should be sufficient such that the binding of phosphorylated BCR-ABL, c-Abl, Bcr, CRKL, Gab1, PYK2, TYK2, SHP2, ERK1/2 and/or MEK1/2 is detectable compared to background.

Antibodies disclosed herein may be conjugated to a solid support suitable for a diagnostic assay (e.g., beads, plates, slides or wells formed from materials such as latex or polystyrene) in accordance with known techniques, such as precipitation. Antibodies of the invention may likewise be conjugated to detectable groups such as radiolabels (e.g., ³⁵S, ¹²⁵I, ¹³¹I), enzyme labels (e.g., horseradish peroxidase, alkaline phosphatase), and fluorescent labels (e.g., fluorescein) in accordance with known techniques. c-Abl, CRKL, Gab1, PYK2, TYK2, SHP2, ERK1/2 and MEK1/2 phospho-specific antibodies of the invention may also be used in a flow cytometry assay to determine the activation status of BCR-ABL in patients before, during, and after treatment with a drug targeted at inhibiting BCR-ABL activity. For example, ficol separated lymphocyte cells from blood samples from CML patients may be analyzed by flow cytometry for BCR-ABL, c-Abl, Bcr, CRKL, Gab1, PYK2, TYK2, SHP2, ERK1/2 and/or MEK1/2 phosphorylation, as well as for markers identifying various hematopoetic cell types. In this manner, BCR-ABL activation status of the malignant cells may be specifically characterized. Flow cytometry may be carried out according to standard methods. See, e.g. Chow et al., Cytometry (Communications in Clinical Cytometry) 46:72-78 (2001). Briefly and by way of example, the following protocol for cytometric analysis may be employed: fixation of the cells with 1% paraformaldehyde for 10 minutes at 37° C. followed by permeabilization in 90% methanol for 30 minutes on ice. Cells may then be stained with the primary c-Abl, Bcr, CRKL, Gab1, PYK2, TYK2, SHP2, ERK1/2 and MEK1/2 antibodies, washed and labeled with fluorescent-labeled secondary antibodies. Alternatively, the primary antibodies may be directly labeled with the fluorescent dye. The cells would then be analyzed on a flow cytometer (e.g. a Beckman Coulter EPICS-XL) according to the specific protocols of the instrument used. Such an analysis would identify the presence of activated BCR-ABL in the malignant cells and reveal the drug response on the targeted BCR-ABL protein.

In certain preferred embodiments of the invention, the cellular sample will be a tumor sample from a cancer patient, for example, a breast cancer patient. In other preferred embodiments, multiple tissue samples are prepared as a tissue microarray for IHC-based staining and analysis. Construction of tissue microarrays is well known in the art (Zhang D. et al. Mod Pathol (2003) January;16(1):79-85).

Phosphorylation status(es) in a cellular sample are examined, in accordance with the methods and kits of the invention, using phospho-specific antibodies in a cellular assay, namely, any assay suitable for detecting in vivo protein activity in a particular cell. Examples of suitable cellular assays include the following preferred assays: immunhisto-chemistry (IHC), flow cytometry (FC), immunofluorescence (IF) (all of which are whole cell or tissue-based staining assays), and capture-and-detection (e.g. ELISA), or reversed phase assays (which are cell-lysate based assays).

As previously discussed, cellular analysis of protein acitivation has many advantages. Methods like IHC and FC are well-used and accepted clinical procedures, and thus are highly-desirable assay formats for clinical and prognostic assays. Cellular assays enable examination of protein activity at the cell or tissue level (as opposed to genetic or protein expression level), including the ability to rapidly analyze multiple sequential tissue slices or cells in parallel. In addition, particular cells having activated proteins can be identified, and can, therefore, be directly compared to normal cells to identify differences in in vivo signaling. Further, protein localization (which plays a significant role in protein function) within a cell may be determined, in addition to phosphorylation status.

Immunohistochemical (IHC) staining using tissues (either diseased (e.g. a tumor biopsy) or normal) may be carried out according to well known techniques. See, e.g., ANTIBODIES: A LABORATORY MANUAL, Chapter 10, Harlow & Lane Eds., Cold Spring Harbor Laboratory (1988). Briefly, paraffin-embedded tissue (e.g. tumor tissue) is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary antibody (i.e. phospho-specific antibodies against signal transduction proteins) and secondary antibody; and finally detecting using ABC avidin/biotin method according to manufacturer's instructions.

Alternatively, the biomarkers may be analyzed in an ELISA or reverse-phase array format. For the ELISA format, a capture antibody for each biomarker is affixed to a solid substrate such as a plastic ELISA plate, nitrocellulose membrane or bead. The patient lysate is incubated with the labeled substrate allowing for the capture of the biomarker proteins to the substrate via the capture antibodies. The substrate is then washed. The captured proteins are then detected using a second antibody specific for each protein. The bound detection antibody may be detected by a labeled secondary antibody or by labeling (fluorescent or enzyme) the detection antibody.

In the reverse phase method, lysates of patient samples are fixed to a solid substrate in predetermined locations. The fixed sample is then incubated with the antibodies. After washing, the bound antibodies are detected by various detection methods such as a secondary detection antibodies or by prelabeling the antibodies with fluorescent labels.

Alternatively, phospho-specific antibodies employed in cellular assays may be optimized for use in other clinically-suitable applications, for example bead-based multiplex-type assays, such as IGEN, Luminex™ and/or Bioplex™ assay formats, or otherwise optimized for antibody arrays formats.

Diagnostic kits for carrying out the methods disclosed above are also provided by the invention. Such kits comprise one or more phospho-specific antibodies of the invention, against one or more biomarkers disclosed herein. In one embodiment, the invention provides a kit for detecting the inhibition of BCR-ABL kinase by an inhibitor, said kit comprising (a) at least one phospho-specific antibody selected from the group consisting of:

(i) a Bcr (Tyr177) phospho-specific antibody;

(ii) a PYK2 (Tyr402) phospho-specific antibody;

(iii) a Tyk2 (Tyr1054/1055) phospho-specific antibody;

(iv) a SHP2 (Tyr580) phospho-specific antibody;

(v) a Gab1 (Tyr627) phospho-specific antibody;

(vi) an ERK1/2 (Thr 202/Tyr204) phospho-specific antibody;

(vii) a MEK1/2 (Ser217/221) phospho-specific antibody;

and, optionally, at least one phospho-specific antibody selected from the group consisting of:

(viii) a BCR-ABL (Tyr245) and/or c-Abl (Tyr245) phospho-specific antibody;

(ix) a BCR-ABL (Thr735) and/or c-Abl (Thr735) phospho-specific antibody;

(x) a CRKL (Tyr207) phospho-specific antibody; and

(b) at least one detectable label suitable for use in a cellular assay to detect antibody-target binding.

In another embodiment, the invention provides a kit for identifying a patient likely to respond to a BCR-ABL kinase inhibitor for the treatment of CML or ALL, said kit comprising (a) at least one phospho-specific antibody selected from the group consisting of:

(i) a Bcr (Tyr177) phospho-specific antibody;

(ii) a PYK2 (Tyr402) phospho-specific antibody;

(iii) a Tyk2 (Tyr1054/1055) phospho-specific antibody;

(iv) a SHP2 (Tyr580) phospho-specific antibody;

(v) a Gab1 (Tyr627) phospho-specific antibody;

(vi) an ERK1/2 (Thr 202/Tyr204) phospho-specific antibody;

(vii) a MEK1/2 (Ser217/221) phospho-specific antibody;

and, optionally, at least one phospho-specific antibody selected from the group consisting of:

(viii) a BCR-ABL (Tyr245) and/or c-Abl (Tyr245) phospho-specific antibody;

(ix) a BCR-ABL (Thr735) and/or c-Abl (Thr735) phospho-specific antibody;

(x) a CRKL (Tyr207) phospho-specific antibody; and

(b) at least one detectable label suitable for use in a cellular assay to detect antibody-target binding.

In certain preferred embodiments, the kits comprise two or more of the antibodies listed in (a)(i)-(vii), while in other preferred embodiments, the kits comprises up to four of the antibodies listed in (a)(i)-(vii), or five or more of the antibodies listed in (a)(i)-(vii).

Diagnostic kits provided by the invention may comprise one or more phospho-specific antibodies of the invention conjugated to a solid support and secondary antibodies conjugated to a detectable group. The reagents may also include ancillary agents such as buffering agents and protein stabilizing agents, e.g., polysaccharides and the like. The diagnostic kit may further include, where necessary, other members of the signal-producing system of which system the detectable group is a member (e.g., enzyme substrates), agents for reducing background interference in a test, control reagents, apparatus for conducting a test, and the like. The test kit may be packaged in any suitable manner, typically with all elements in a single container along with a sheet of printed instructions for carrying out the test.

Such kits enable the detection of BCR-ABL kinase, c-Abl kinase, and Abl signaling pathway activation by sensitive cellular assay methods, such as IHC and flow cytometry, which are suitable for the clinical detection, prognosis, and screening of cells and tissue from patients, such as leukemia patients, having a disease involving altered c-Abl pathway signaling.

The following examples are provided only to further illustrate the invention, and are not intended to limit its scope, except as provided in the claims appended hereto. The present invention encompasses modifications and variations of the methods taught herein which would be obvious to one of ordinary skill in the art.

### EXAMPLE 1

### Identification of Biomarkers of BCR-ABL Inhibition in CML Tissue

Western blot analysis of lysates of K562 cells (a CML cell line) before and after treatment with the BCR-ABL inhibitor, Gleevec®, were performed in order to identify relevant biomarkers for inhibition. Lysates were analyzed with phospho-specific antibodies of the invention against c-Abl, CRKL, Gab1, PYK2, TYK2, SHP2, ERK1/2 and MEK1/2. For the Western blot analysis, K562 cells were obtained from the ATCC.

K562 cells were cultured in DMEM supplemented with 10% FCS. The cells were treated with Gleevec® (10 uM) for 1 hour. The cells were collected, washed with PBS and directly lysed in cell lysis buffer. The protein concentrations of cell lysates were measured. The loading buffer was added into cell lysate and the mixture was boiled at 100° C. for 5 minutes. The 20 μl (10 μg protein) of sample was added onto 7.5% SDS-PAGE gel.

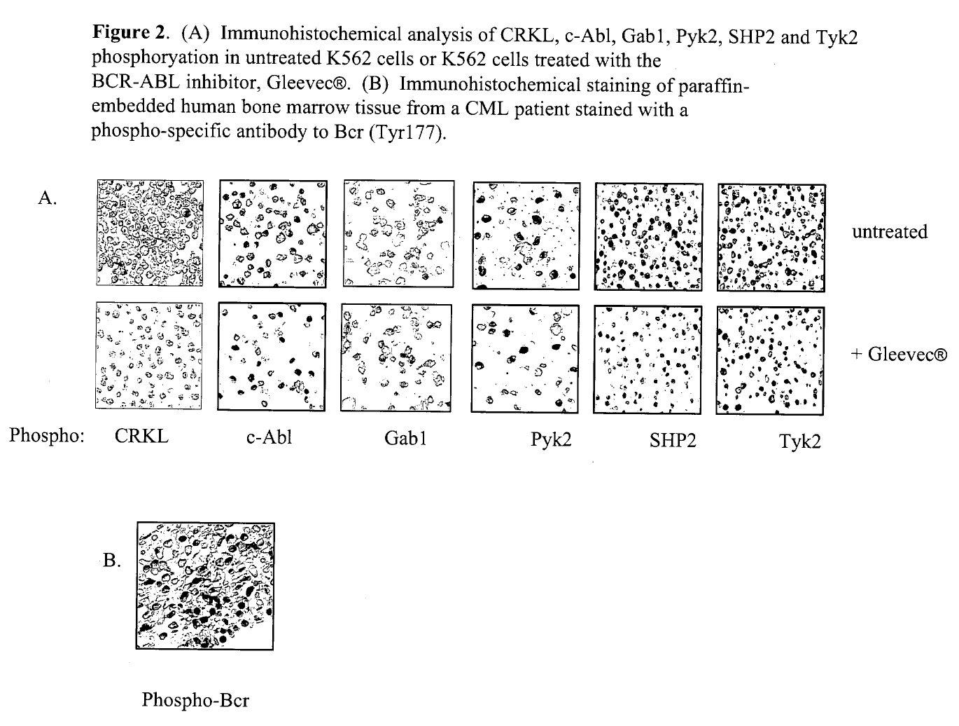

The standard Western blot was performed according to the Immunoblotting Protocol set out in the Cell Signaling Technology, Inc. 2002 Catalogue and Technical Reference, p. 282. The antibodies were used at a 1:1000 dilution. The results of the blots are shown in FIG. 2.

As shown in the Figure, the antibodies recognize only a protein with a molecular weight predicted for that target. In addition, the intensity of the signal for each protein decreases in the Gleevec® treated samples. These results demonstrate that these antibodies are capable of detecting a decrease in phosphorylation of these proteins following inhibition of BCR-ABL. Therefore, these biomarkers and corresponding antibodies may be used as biomarkers of Gleevec®) treatment.

### EXAMPLE 2

### Detection of Biomarker Phosphorylation in CML Tissue by Immunohistochemical Analysis

Immunohistochemical (IHC) analysis of paraffin-embedded samples is the most common method for analyzing the pathology of diseased tissues. Determining the molecular pathology of a tumor may also be obtained through an IHC analysis of paraffin-embedded tissues. New cancer therapies targeted at the BCR-ABL require that the patient have active BCR-ABL and that downstream signaling is active as well. Therefore, phospho-specific antibodies to downstream signaling molecules may be used to prescreen patients for inclusion in a clinical trial, to follow patients during treatment and to detect resistance to the targeted therapeutic.

For IHC analysis, custom tissue microarrays of human bone marrow (obtained by standard biopsy procedures from a CML patient followed by fixation of the tissue in formalin) were commercially obtained (Clinomics, Inc.). The tissue in the arrays was paraffin-embedded following standard procedures (see ANTIBODIES, A LABORATORY MANUAL, supra.).

Alternatively, K562 chronic myelogenous leukemia cells were grown in cell culture and treated with Gleevec®. The cells were then washed, spun down and the cell pellet was fixed and embedded in paraffin. For IHC staining, 2-4 micron thick slices were cut from the paraffin blocks using a microtome and placed on glass slides. The sections were then de-paraffinized with xylene and ethanol, then microwaved for 10 minutes in a sodium citrate pH 6.0 or EDTA pH 8.0 buffer for antigen retrieval. After a 10 minute incubation in 3% H2O2, the sections were blocked in 5% goat serum for 1 hour.

The slides were then stained with SHP2, Tyk2, PYK2, CRKL, c-Abl and Gab1 phospho-antibodies for 2 hours at room temperature or overnight at 4C. After 3 washes in PBS or TBS with 0.1% Tween 20, the slides were then probed with a secondary antibody labeled with biotin. The slides are further developed with a avidin-biotin-HRP reagent (ABC kit) following standard manufacturer procedures. The slides were developed using a HRP substrate, either DAB or NovaRed™ and counterstained with hematoxylin. Positive staining for antibody staining was scored based upon staining intensity, number of cells stained and correct localization of stain: See FIG. 2.

An alternative method for analyzing patient blood samples commonly used in the clinic is IHC analysis of cell smears. In this procedure patient blood samples from a normal healthy patient and a patient with Gleevec®) resistant CML were ficoll-separated to isolate lymphocytes which were then prepared as described below in Example 3. The fixed lymphocytes were then spotted onto glass slides, allowed to dry and then stained as described above for paraffin-embedded tissues. The slides were stained with phospho-antibodies against CRKL and c-Abl.

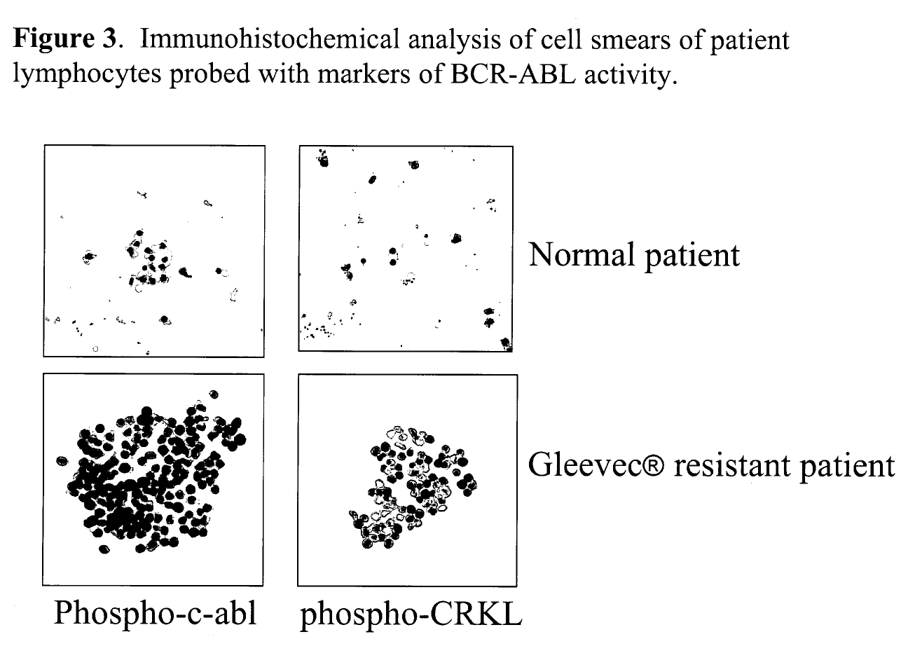

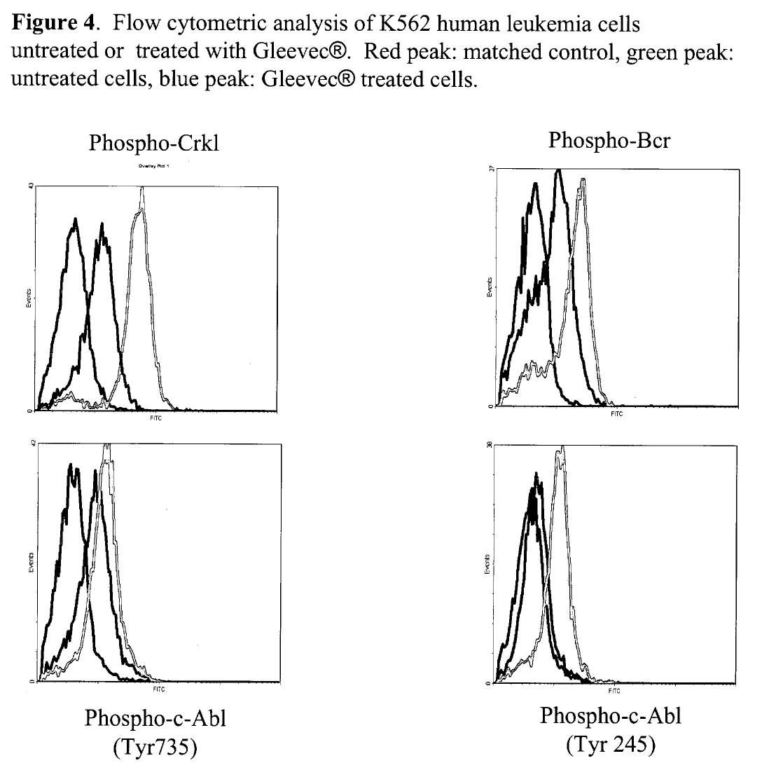

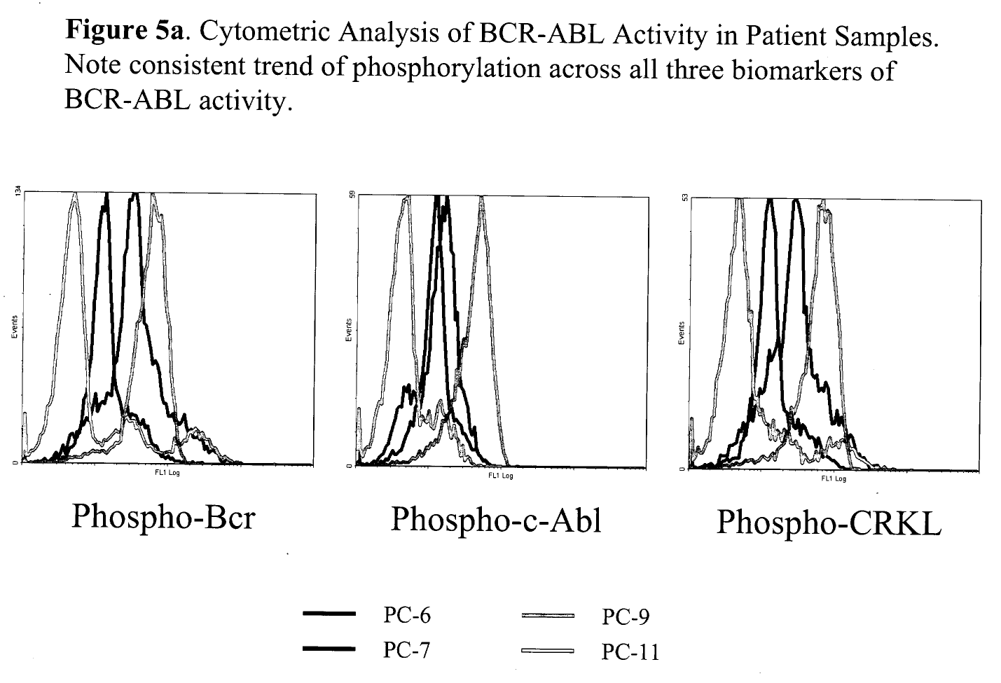

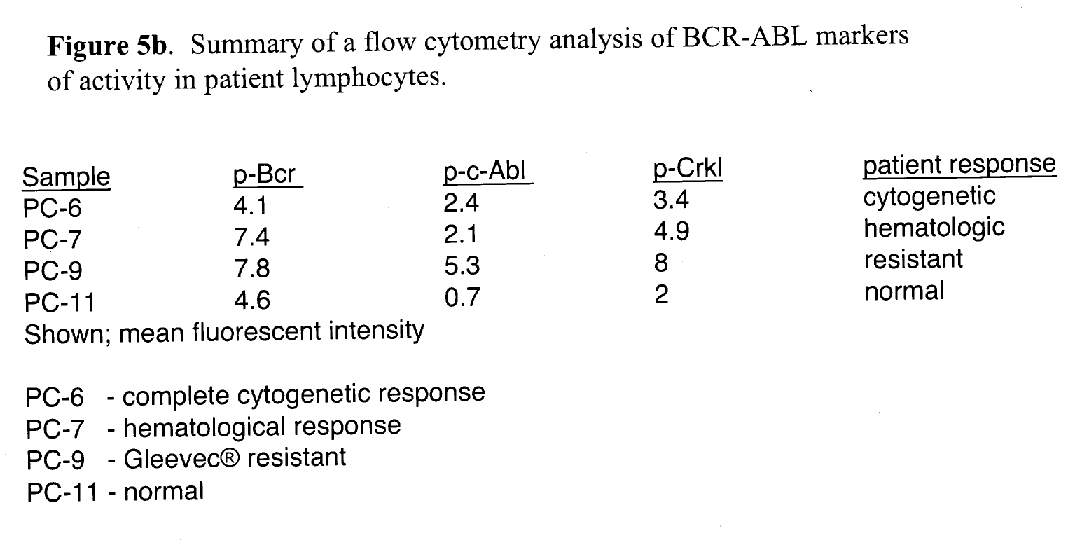

The results (FIG. 3) clearly demonstrate strong staining in the Gleevec® resistant cells, but no staining in the normal lymphocytes. This observation is consistent with restored BCR-ABL activity in Gleevec® resistant leukemia cells.

### EXAMPLE 3

### Flow Cytometric Analysis of CML Patient Samples Using Panels of Phospho-Specific Antibodies

c-Abl, CRKL and Bcr phospho-specific antibodies were used in flow cytometry to detect phosphorylated proteins in K562 cells and human lymphocytes with and without treatment with Gleevec®). K562 cells were incubated with or without the Gleevec® (2 uM) for 1 hour at 37° C. The cells then were fixed with 2% paraformaldehyde for 20 minutes at 37° C. followed by cell permeabilization 90% with methanol for 20 minutes on ice. The fixed cells were then stained with the primary antibodies for 30 minutes at room temperature. After incubation with a FITC-conjugated secondary antibody, the cells were analyzed on a Beckman Coulter EPICS-XL flow cytometer. The cytometric results with K562 cells demonstrate the specificity of the antibodies for the detection of Gleevec® treatment as the peak in the treated population is always significantly to the left of the peak shown for the untreated cell population (FIG. 3A).

Fresh CML patient blood samples were obtained from Dr. Charles Sawyers at UCLA under an approved IRB. The blood samples were separated on a ficol gradient yielding a lymphocyte cell population. The cells were then fixed as described above for the K562 cells. The fixed cells were stained with the c-Abl, Bcr (Tyr177) and CRKL phospho-specific antibodies, FITC-conjugated secondaries and analyzed as described.

The results are from the entire cell population and are the analysis was not gated on a subpopulation of cells. Patient #6 was on Gleevec® and showed a complete cytogenetic response. Patient #9 was also on Gleevec® but was showing resistance with a large over-proliferation of leukemia cells. Patient #11 did not have leukemia and had normal lymphocytes. The analysis clearly shows that the resistant patient (#9) has a much larger amount of phosphorylation of the three proteins compared to the other to samples. This result is consistent with effective Gleevec® inhibition of BCR-ABL in patient #6 returning the phosphorylation levels to those observed in normal lymphocytes (patient #11). This analysis demonstrates a potential clinical assay that may be used to follow patient response to Gleevec® treatment and the detection of Gleevec® resistance.

TABLE 1
Phosphorylation-specific antibodies for
the detection of BCR-ABL activity.

| Protein | Phospho-residue |
|---|---|
| c-Abl (BCR-ABL) | Tyr245 |
| c-Abl (BCR-ABL) | Tyr735 |
| Bcr (BCR-ABL) | Tyr177 |
| CRKL | Tyr207 |
| Gab1 | Tyr627 |
| PYK2 | Tyr402 |
| Tyk2 | Tyr1054/1055 |
| SHP2 | Tyr580 |
| MEK1/2 | Ser217/221 |
| ERK1/2 | Thr202/Tyr204 |

What is claimed is:

1. A method for detecting the activity of BCR-ABL or c-Abl kinase and/or the c-Abl signaling pathway in a cell or tissue, said method comprising the steps of:
(a) obtaining at least one test cell or tissue from a subject;
(b) contacting said test cell or tissue with at least one phospho-specific antibody selected from the group consisting of:
(i) a Bcr (Tyr177) phospho-specific antibody;
(ii) a PYK2 (Tyr402) phospho-specific antibody;
(iii) a Tyk2 (Tyr1054/1055) phospho-specific antibody;
(iv) a SHP2 (Tyr580) phospho-specific antibody;
(v) a Gab1 (Tyr627) phospho-specific antibody;
(vi) an ERK1/2 (Thr 202/Tyr204) phospho-specific antibody;
(vii) a MEK1/2 (Ser217/221) phospho-specific antibody;
and, optionally, at least one phospho-specific antibody selected from the group consisting of:
(viii) a BCR-ABL (Tyr245) and/or c-Abl (Tyr245) phospho-specific antibody;
(ix) a BCR-ABL (Thr735) and/or c-Abl (Thr735) phospho-specific antibody;
(x) a CRKL (Tyr207) phospho-specific antibody;
(c) determining the level of at least one of phosphorylated c-Abl, Bcr, Gab1, PYK2, TYK2, SHP2, ERK1/2 and/or MEK1/2, and optionally, at least one of BCR-ABL or CRKL, bound by the antibody of step (b); and
(d) comparing the level of phosphorylated protein determined in step(c) for said test cell or tissue with the level of phosphorylated protein in a reference sample, thereby detecting the activity of BCR-ABL, c-Abl, and/or the c-Abl signaling pathway in said test cell or tissue.

2. The method of claim 1, wherein said subject has, or is at risk of, cancer.

3. The method of claim 2, wherein said cancer is chronic myelogenous leukemia (CML) or acute lymphocytic leukemia (ALL).

4. The method of claim 2, wherein the determination of phosphorylated protein levels in step (b) comprises conducting immunohistochemistry (IHC) and/or flow cytometry.

5. The method of claim 1, wherein step (b) comprises contacting said test cell or tissue with two or more of said phospho-specific antibodies listed in (i)-(vii) of step (b).

6. A method for detecting the inhibition of BCR-ABL or c-Abl kinase by an inhibitor, said method comprising the steps of:
(a) obtaining at least one test cell or tissue from a subject;
(b) contacting said test cell or tissue with said inhibitor and at least one phospho-specific antibody selected from the group consisting of:
(i) a Bcr (Tyr177) phospho-specific antibody;
(ii) a PYK2 (Tyr402) phospho-specific antibody;
(iii) a Tyk2 (Tyr1054/1055) phospho-specific antibody;
(iv) a SHP2 (Tyr580) phospho-specific antibody;
(v) a Gab1 (Tyr627) phospho-specific antibody;
(vi) an ERK1/2 (Thr 202/Tyr204) phospho-specific antibody;
(vii) a MEK1/2 (Ser217/221) phospho-specific antibody;
and, optionally, at least one phospho-specific antibody selected from the group consisting of:
(viii) a BCR-ABL (Tyr245) and/or c-Abl (Tyr245) phospho-specific antibody;
(ix) a BCR-ABL (Thr735) and/or c-Abl (Thr735) phospho-specific antibody;
(x) a CRKL (Tyr207) phospho-specific antibody;
(c) conducting a cellular assay with said test cell or tissue to determine the level of at least one of phosphorylated c-Abl, Bcr, Gab1, PYK2, Tyk2, SHP2, ERK1/2 and/or MEK1/2, and optionally at least one of BCR-ABL or CRKL, bound by the antibody of step (b); and
(d) comparing the level of phosphorylated protein determined in step(c) for said test cell or tissue with the level of phosphorylated protein in a reference sample not treated with said inhibitor, thereby detecting the inhibition of BCR-ABL or c-Abl kinase by said inhibitor in said test cell or tissue.

7. A method for identifying a patient likely to respond to a BCR-ABL kinase inhibitor for the treatment of CML or ALL, said method comprising the steps of:
(a) obtaining at least one test cell or tissue from a patient having CML or ALL;
(b) contacting said test cell or tissue with at least one phospho-specific antibody selected from the group consisting of:
(i) a Bcr (Tyr177) phospho-specific antibody;
(ii) a PYK2 (Tyr402) phospho-specific antibody;
(iii) a Tyk2 (Tyr1054/1055) phospho-specific antibody;
(iv) a SHP2 (Tyr580) phospho-specific antibody;
(v) a Gab1 (Tyr627) phospho-specific antibody;
(vi) an ERK1/2 (Thr 202/Tyr204) phospho-specific antibody;
(vii) a MEK1/2 (Ser217/221) phospho-specific antibody;
and, optionally, at least one phospho-specific antibody selected from the group consisting of:
(viii) a BCR-ABL (Tyr245) and/or c-Abl (Tyr245) phospho-specific antibody;
(ix) a BCR-ABL (Thr735) and/or c-Abl (Thr735) phospho-specific antibody;
(x) a CRKL (Tyr207) phospho-specific antibody;
(c) conducting a cellular assay with said test cell or tissue to determine the level of at least one of phosphorylated c-Abl, Bcr, Gab1, PYK2, Tyk2, SHP2, ERK1/2 and/or MEK1/2, and optionally at least one of phosphorylaetd BCR-ABL or CRKL, bound by the antibody of step (b), wherein a significantly high level of one or more of these phosphorylated proteins identifies a patient likely to respond to a BCR-ABL kinase inhibitor for the treatment of CML or ALL.

8. The method of claim 8, further comprising the step of (d) comparing the level of phosphorylated protein determined in step(c) for said test cell or tissue with the level of phosphorylated protein in a reference sample characteristic of CML or ALL patients responsive to a BCR-ABL inhibitor.

9. The method of claims 6 or 7, wherein the cellular assay of step (c) comprises conducting immunohistochemistry (IHC) and/or flow cytometry.

10. The method of claims 6 or 7, wherein step (b) comprises contacting said test cell or tissue with two or more of said phospho-specific antibodies listed in (i)-(vii) of step (b).

11. The method of claims 6 or 7, wherein step (b) comprises contacting said test cell or tissue with three or more phospho-specific antibodies comprising said Bcr (Tyr177) phospho-specific antibody, said c-Abl (Tyr245) and/or (Thr735) phospho-specific antibody, and said CRKL (Tyr207) phospho-specific antibody.

12. A method for identifying one or more protein biomarker(s) of patient response or resistance to a BCR-ABL inhibitor for the treatment of CML or ALL, said method comprising the steps of:
(a) obtaining at least one test cell or tissue from (i) each of a plurality of BCR-ABL inhibitor-responsive patients having CML or ALL, (ii) each of a plurality of BCR-ABL inhibitor-resistant patients having CML or ALL, and (iii) control patients having neither disease;
(b) contacting said test cells or tissues with two or more phospho-specific antibodies selected from group consisting of:
(i) a Bcr (Tyr177) phospho-specific antibody;
(ii) a PYK2 (Tyr402) phospho-specific antibody;
(iii) a Tyk2 (Tyr1054/1055) phospho-specific antibody;
(iv) a SHP2 (Tyr580) phospho-specific antibody;
(v) a Gab1 (Tyr627) phospho-specific antibody;
(vi) an ERK1/2 (Thr 202/Tyr204) phospho-specific antibody;
(vii) a MEK1/2 (Ser217/221) phospho-specific antibody;
and, optionally, at least one phospho-specific antibody selected from the group consisting of:
(viii) a BCR-ABL (Tyr245) and/or c-Abl (Tyr245) phospho-specific antibody;
(ix) a BCR-ABL (Thr735) and/or c-Abl (Thr735) phospho-specific antibody;
(x) a CRKL (Tyr207) phospho-specific antibody;
(c) conducting a cellular assay with said test cells or tissues to determine the level of two or more of phosphorylated c-Abl, Bcr, Gab1, PYK2, Tyk2, SHP2, ERK1/2 and/or MEK1/2, and optionally at least one of phosphorylated BCR-ABL or CRKL, bound by the antibodies of step (b), thereby generating an activation profile for said inhibitor-responsive and inhibitor-resistant patients and said control patients; and
(d) comparing said activation profiles of step (c), whereby a substantial difference in the activation profiles for said inhibitor-responsive and said inhibitor-resistant patients as compared to said control patients identifies one or more signal transduction protein(s) as being associated with patient responsiveness or resistance to a BCR-ABL inhibitor for the treatment of CML or ALL.

13. The method of any one of claims 6, 7, or 12, wherein said inhibitor comprises an ABL kinase inhibitor.

14. The method of claim 13, wherein said ABL kinase inhibitor is Gleevec (STI-571).

15. A kit for detecting the inhibition of BCR-ABL kinase by an inhibitor, said kit comprising (a) at least one phospho-specific antibody selected from the group consisting of:
(i) a Bcr (Tyr177) phospho-specific antibody;
(ii) a PYK2 (Tyr402) phospho-specific antibody;
(iii) a Tyk2 (Tyr1054/1055) phospho-specific antibody;
(iv) a SHP2 (Tyr580) phospho-specific antibody;
(v) a Gab1 (Tyr627) phospho-specific antibody;
(vi) an ERK1/2 (Thr 202/Tyr204) phospho-specific antibody;
(vii) a MEK1/2 (Ser217/221) phospho-specific antibody;
and, optionally, at least one phospho-specific antibody selected from the group consisting of:
(viii) a BCR-ABL (Tyr245) and/or c-Abl (Tyr245) phospho-specific antibody;
(ix) a BCR-ABL (Thr735) and/or c-Abl (Thr735) phospho-specific antibody;
(x) a CRKL (Tyr207) phospho-specific antibody; and
(b) at least one detectable label suitable for use in a cellular assay to detect antibody-target binding.

16. A kit for identifying a patient likely to respond to a BCR-ABL kinase inhibitor for the treatment of CML or ALL, said kit comprising (a) at least one phospho-specific antibody selected from the group consisting of:
(i) a Bcr (Tyr177) phospho-specific antibody;
(ii) a PYK2 (Tyr402) phospho-specific antibody;
(iii) a Tyk2 (Tyr1054/1055) phospho-specific antibody;
(iv) a SHP2 (Tyr580) phospho-specific antibody;
(v) a Gab1 (Tyr627) phospho-specific antibody;
(vi) an ERK1/2 (Thr 202/Tyr204) phospho-specific antibody;
(vii) a MEK1/2 (Ser217/221) phospho-specific antibody;
and, optionally, at least one phospho-specific antibody selected from the group consisting of:
(viii) a BCR-ABL (Tyr245) and/or c-Abl (Tyr245) phospho-specific antibody;
(ix) a BCR-ABL (Thr735) and/or c-Abl (Thr735) phospho-specific antibody;
(x) a CRKL (Tyr207) phospho-specific antibody; and
(b) at least one detectable label suitable for use in a cellular assay to detect antibody-target binding.

17. The kit of claim 15 or 16, wherein said kit comprises two or more of the antibodies listed in (a)(i)-(vii).

18. The kit of claim 15 or 16, wherein said kit comprises up to four of the antibodies listed in (a)(i)-(vii).

19. The kit of claim 15 or 16, wherein said kit comprises five or more of the antibodies listed in (a)(i)-(vii).
